(19) United States
(12) Patent Application Publication
(10) Pub. No.: US 2004/0091538 A1
(43) Pub. Date: May 13, 2004

(54) Dosage form providing ascending release of liquid formulation

(76) Inventors: Crystal Pollock-Dove, Mountain View, CA (US); Liang Dong, Sunnyvale, CA (US); Patrick S. L. Wong, Burlingame, CA (US); Si-Hong Yum, Belmont, CA (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/700,941

(22) Filed: Oct. 31, 2003

Related U.S. Application Data
(60) Provisional application No. 60/423,099, filed on Oct. 31, 2002.

(51) Int. Cl. A61K 9/24

(52) U.S. Cl. 424/473

(57) ABSTRACT

The present invention includes a dosage form that releases a liquid active agent formulation over a period of time at an ascending rate. The dosage form of the present invention includes a capsule or other reservoir capable of containing a liquid active agent formulation, a driving means for expelling the liquid active agent formulation from the capsule over an extended period of time and a rate altering means for increasing the rate at which the driving means expels liquid active agent formulation from the capsule. The present invention also includes a method of manufacturing a controlled release dosage form providing the release of liquid active agent formulation at an ascending rate. The method of the present invention includes providing a capsule or reservoir suitable for containing a liquid active agent formulation, filling the capsule with a liquid active agent formulation, providing the capsule with a driving means for expelling the liquid active agent formulation from the capsule to an environment of use, and providing a rate altering means for increasing the rate at which the driving means expels the liquid active agent formulation.

### BACKGROUND

This application claims the benefit of U.S. Provisional Application No. 60/423,099, filed Oct. 31, 2002.

### FIELD OF THE INVENTION

The present invention includes a dosage form providing an ascending release of a liquid formulation. In particular, the present invention includes a dosage for delivering a liquid formulation that includes a membrane exhibiting a permeability that increases over time, which facilitates delivery of the liquid formulation at an ascending rate.

### STATE OF THE ART

Dosage forms providing the controlled release of a liquid formulation are known in the art. For example, U.S. Pat. Nos. 6,419,952, 6,342,249, 6,183,466, 6,174,547, 5,614,578, 5,413,572, 5,324,280, and 4,627,850 assigned to ALZA corporation, which are herein incorporated by this reference, teach various different dosage forms providing controlled release of a liquid formulation. The dosage forms described in these references generally include a hard or soft capsule for containing the liquid formulation, an osmotic composition, a semipermeable outer membrane, and an exit orifice. As aqueous fluid from an environment of use is absorbed into the osmotic composition included in these dosage forms, the osmotic composition expands and drives the liquid formulation from the dosage form through the exit orifice. Generally, the material make-up and thickness of the semipermeable membrane included in a controlled release osmotic dosage form for the delivery of a liquid formulation controls the rate at which aqueous fluid enters the dosage form and hydrates the osmotic composition. Therefore, the semipermeabile membrane of a controlled release osmotic dosage form for the delivery of a liquid formulation can be modified to provide a desired release rate.

Various active agents or active agent formulations, however, may benefit from controlled release within the gastrointestinal (“GI”) tract of a subject at an ascending rate. For instance, various active agents may provide increased therapeutic value or decreased side effects when delivered at an ascending rate over time within the GI tract of a subject. Moreover, active agent formulations may facilitate increased bioavailability of the active agents contained therein when released at an ascending rate from a controlled release dosage form. For example, when compared to the upper GI tract, the environmental conditions in the lower portions of the GI tract of a subject, such as the relatively higher pH, the presence or absence of particular enzymes, or the relatively smaller amount of aqueous media, may be more conducive to the GI absorption of an active agent from a particular active agent formulation. Where an active agent exhibits increased bioavailability in the lower GI tract or where a particular active agent formulation allows increased absorption of active agent when delivered to the lower GI tract, a dosage form that delivers an active agent formulation over time at an ascending rate may better assure that relatively more active agent formulation is delivered to the lower portions of the GI tract, where the active agent will be more readily absorbed. Therefore, it would be desirable to provide a dosage form capable of delivering a liquid active agent formulation over a desired period of time at an ascending rate. In particular, it would be desirable if such a dosage form were capable of delivering a variety of different active agents in a variety of different liquid formulations at an ascending rate within a desired area in the GI tract of a subject.

### SUMMARY OF THE INVENTION

The present invention includes a dosage form that releases a liquid active agent formulation over a period of time at an ascending rate. The dosage form of the present invention includes a capsule or other reservoir capable of containing a liquid active agent formulation, a driving means for expelling the liquid active agent formulation from the capsule over an extended period of time and a rate altering means for increasing the rate at which the driving means expels liquid active agent formulation from the capsule. In one aspect, the dosage form of the present invention includes an osmotic dosage form that is formed using hard or soft capsule. An osmotic dosage form according to the present invention includes an expandable osmotic composition that works to expel liquid active agent formulation from the capsule upon hydration, semipermeable membrane that allows hydration of the osmotic composition but is impermeable to active agent material, and an ascending release material positioned between the semipermeable membrane and the osmotic composition. The ascending release material included in an osmotic dosage form of the present invention causes the rate of hydration of the osmotic composition to increase over time, which, in-turn, causes the osmotic layer to expand at an increasing rate and results in an ascending rate of release of liquid active agent formulation from the dosage form. The dosage form of the present invention is suitable for delivering a wide range of liquid active agent formulations to an environment of use.

The present invention also includes a method of manufacturing a controlled release dosage form providing the release of liquid active agent formulation at an ascending rate. The method of the present invention includes providing a capsule or reservoir suitable for containing a liquid active agent formulation, filling the capsule with a liquid active agent formulation, providing the capsule with a driving means for expelling the liquid active agent formulation from the capsule to an environment of use, and providing a rate altering means for increasing the rate at which the driving means expels the liquid active agent formulation. In one embodiment, the method of the present invention includes providing a capsule, loading the capsule with a liquid active agent formulation, providing the capsule with an expandable osmotic composition, and providing the capsule with an ascending release material such that the rate of hydration of the osmotic composition increases over time when the dosage form delivered to an environment of use. Of course, the method of the present invention may be altered as desired to achieve a dosage form that delivers a liquid active agent formulation over a targeted period of time at an desired ascending rate.

### BRIEF DESCRIPTION OF THE DRAWINGS

FIG. 1 provides a schematic cross-sectional representation of a soft-cap ascending release dosage form according to the present invention.

FIG. 2 and FIG. 3 provide schematic cross-section representations of two different hard-cap ascending release dosage forms according to the present invention.

FIG. 4. provides a graph illustrating the ascending release rate profiles provided by hard-cap ascending release dosage forms prepared according to the present invention.

FIG. 5 provides a graph illustrating the release rate profile of a soft-cap ascending release dosage form prepared according to the present invention.

### DETAILED DESCRIPTION OF THE INVENTION

A dosage form of the present invention includes a dosage form providing the release of a liquid active agent formulation to an environment of operation at an ascending rate over an extended period of time. As they are used herein, the terms “ascending rate” and “ascending release rate” indicate a rate of release of liquid active agent formulation from a dosage form that increases over a period of hours. In particular, the terms “ascending rate” and “ascending release rate” refer to a rate of release of liquid active agent formulation that increases over a period of about 2 hours or greater, with periods of about 2 hours to about 24 hours being preferred, and a periods of about 4 hours to about 12 hours being particularly preferred. As it is used herein, the term “environment of operation” refers to an environment containing water or water containing fluids, including in vivo media found in animals, such as the aqueous fluid present in the GI tract of an animal.

The dosage form of the present invention includes a capsule or other reservoir suitable for containing the liquid active agent formulation. The dosage form is further provided with a driving means that serves to expel the liquid active agent formulation from the capsule after the dosage form has been delivered to an environment of operation. Importantly, the dosage form of the present invention also includes a rate altering means, which serves to increase the rate at which the driving means expels the liquid active agent formulation from the capsule. The dosage form of the present invention may include any capsule or reservoir that may be used to deliver a desired liquid active agent formulation, and the driving means may constitute any material or mechanism that allows expulsion of the liquid active agent formulation from the capsule at an ascending rate over a desired period of time after the dosage form has been delivered to an environment of use. The rate altering means may also include any material or mechanism capable of increasing the rate at which the driving means expels the liquid active agent formulation from the capsule such that the liquid active agent formulation is released into the environment of use at an ascending rate.

In one embodiment, the dosage form of the present invention is an osmotic dosage form. An osmotic dosage form of the present invention will generally include a capsule filled with a liquid active agent formulation, and driving means formed by an expandable osmotic composition, a semipermeable membrane providing structural support for the dosage form and allowing controlled hydration of the osmotic composition, and a rate altering means provided by an ascending release material positioned such that the rate of hydration of the osmotic composition increases and the osmotic composition expels the liquid active agent formulation from the dosage form at an ascending release rate. The ascending release material included in an osmotic dosage form of the present invention exhibits a permeability that increases with time after the dosage form has been placed in an environment of operation. As the permeability of the ascending release material increases the rate at which aqueous fluid can flow into the osmotic composition increases, causing the osmotic composition to hydrate at an increasing rate over time. As the osmotic composition hydrates at an increasing rate, the osmotic composition expands at an increasing rate and provides an ascending release rate of liquid active agent formulation from the dosage form of the present invention.

The ascending release material included in an osmotic dosage form of the present invention will generally be placed adjacent to the semipermeable membrane. As it is used herein, the term “adjacent” indicates that the ascending release material is positioned over or under the semipermeable membrane but not necessarily in direct contract with the semipermeable membrane. For example, the ascending release material may be positioned immediately over or immediately under the semipermeable membrane. Alternatively, the ascending release material may be separated from the semipermeable membrane by one or more additional material layers. However, in order to provide ascending release of the liquid active agent formulation, the ascending release material must be positioned such that aqueous fluid from the environment of use flows through the ascending release material before it reaches the osmotic composition included in an osmotic dosage form of the present invention.

The ascending release material included in an osmotic dosage form according to the present invention may be any material that can be provided in or on a dosage form and exhibits an increasing permeability over time in an environment of operation. In one embodiment, the ascending release material is formed using a polymer membrane that exhibits a permeability that increases with time in an environment of use. In one embodiment, an ascending release polymer membrane according to the present invention is formed of a hydrophobic polymer material and a swellable hydrophilic material. The swellable hydrophilic material may include any material that may be blended into a polymer membrane and swells as it hydrates in an environment of operation. In a preferred embodiment, the swellable hydrophilic material is a swellable hydrophilic polymer. As it is used herein, the term “ascending release membrane” is interchangeable with the term “ascending release material.”

Where the ascending release membrane according to the present invention is formed of hydrophobic polymer material and a swellable hydrophilic material, the ascending release membrane is formulated to exhibit a relatively low initial permeability. The low initial permeability of the ascending release membrane results in a relatively low initial rate of hydration of the osmotic composition included in the dosage form. As aqueous fluid passes through an ascending release membrane formed of a hydrophobic polymer and a swellable hydrophilic material, however, the swellable hydrophilic material absorbs water and expands. Over a period of time, the swelling of the hydrophilic material is believed to create channels that allow water to more readily flow through the ascending release membrane and thereby increase the water permeability of the ascending release membrane as a function of time. In particular it is believed, that over time, particles of the swellable hydrophilic material swell and come into contact with other swellable hydrophilic particles and that the contacting particles form channels through which water flows through the ascending release membrane. The relative amounts of hydrophobic polymer and swellable hydrophilic materials included in a polymer ascending release membrane according to the present invention can be varied to provide an ascending release membrane exhibiting a targeted change in permeability or a desired ascending release rate.

Polymer materials suitable for forming an ascending release membrane included in an osmotic dosage form of the present invention include any polymer material or combination of polymer materials that provide a pharmaceutically acceptable ascending release membrane that exhibits a permeability that increases over time in a desired environment of operation. However, the hydrophobic polymer material preferably allows the coating of a flexible membrane that allows the swelling of the swellable hydrophilic substance without compromising the membrane and the swellable hydrophilic substance is preferably chosen such that it swells within the membrane but does not elute or dissolve out of the membrane, at least until after a desired release profile has been achieved. Where the membrane is formed of a flexible hydrophobic polymer and the swellable hydrophilic material dissolves or elutes out of the membrane, the hydrophobic polymer may flow to close up the vacancies left by the hydrophilic material and thereby reduce the permeability of the membrane. An ascending release polymer membrane formed according to the present invention will include about 80 wt % to about 50 wt % hydrophobic polymer material and about 20 wt % to about 50 wt % swellable hydrophilic material, with polymer membranes including about 60 wt % to about 70 wt % hydrophobic polymer material and about 30 wt % to about 40 wt % swellable hydrophilic material being preferred.

Though various combinations of hydrophobic polymer material and swellable hydrophilic material may be used to form an ascending release membrane according to the present invention, membranes formed using blends of acrylic and vinyl polymers have been found to produce flexible ascending release membranes providing desirable release rate characteristics. Acrylic polymer materials that may serve as the hydrophobic portion of an ascending release membrane according to the present invention include Eudragit NE and Eudragit FS. In particular, an 85/15 wt/wt blend of Eudragit NE/Eudragit FS combined with a cross linked polyvinylpyrrolidone has been found to provide an ascending release membrane exhibiting desirable flexibility and release rate characteristics. The 85/15 blend of Eudragit NE and Eudragit FS allows the coating of a uniform ascending release membrane using standard coating techniques that require little or no glidant material. Alone, Eudragit NE provides a suitably hydrophobic coating, but the coating is tacky and requires the use of a relatively large amount of glidant to prevent aggregation of dosage forms during and after the coating process. Blending Eudragit FS with Eudragit NE provides a hydrophobic coating that is still suitably flexible, but does not exhibit the tackiness of Eudragit NE alone and can be coated onto a dosage form using standard spray coating techniques that utilize relatively little or no glidant material.

The ascending release membrane included in an osmotic dosage form according to the present invention is not limited to a membrane formed by a blend of Eudragit NE and Eudragit FS combined with a cross linked polyvinylpyrrolidone. Additional exemplary hydrophobic polymers that may be suitable for formation of an ascending release membrane according to the present invention include polystyrene, polyamides, polyvinyl acetate, poly-methylmethacrylate, ethyl acrylate methyl methacrylate copolymer, ethyl acrylate methyl methacrylate copolymer, poly(butyl methacrylate (2-dimethyl aminoethyl)methacrylate, methyl methacrylate), methacrylic acid methylmethacrylate copolymer, and the like. Additional swellable hydrophilic materials that may be suitable for formation of an ascending release membrane according to the present invention include, for example, low substituted hydroxypropyl cellulose, hydroxypropyl methylcellulose, methylcellulose, hydroxyethyl methylcellulose, polyvinyl acetate polyvinyl pyrrolidone copolymer, gelatin, starch, polyethylene glycol polyvinyl alcohol copolymer, carrageenan, algin, agar, gum acacia, gum karyara, carob bean gum, gum tragacanth, gum ghatti guar gum, caseinates, cellulose acetate with an acetyl content of less than 20 wt %, sodium carboxymethyl cellulose, potassium carboxy methyl cellulose, polyvinyl alcohol, polyvinyl alcohol polyethylene glycol graph copolymers, cellulose acetate phthalate, hydroxypropyl methycellulose phthalate, hydroxypropyl methyl cellulose acetate succinate, or any blends, molecular weights, or combinations of each, as desired. An ascending release membrane according to the present invention may also be formulated using more than one different hydrophobic polymer or more than one different swellable hydrophilic substance.

The ascending release material or membrane included in a dosage form according to the present invention may be provided on the dosage form using any suitable process. For example, where the ascending release material is formed of a material that can be coated, a dosage form of the present invention may be provided with a desired coating of the ascending release material using any suitable spray coating or dip coating techniques. Alternatively, the ascending release material may be compressed in a desired shape around an intermediate dosage form assembly, or the ascending release material may be formed into a desired shape and then bonded to an intermediate dosage form assembly using a water permeable and biologically compatible adhesive. As it is used herein, the term “intermediate dosage form assembly” indicates an assembly that includes one or more components of a dosage form of the present invention, but does not yet include every component of a dosage form of the present invention.

An ascending release dosage form of the present invention may be provided with any desired liquid active agent formulation. As it used herein, the expression “active agent” encompasses any drug, therapeutic compound, or composition that can be delivered to provide a benefit to an intended subject. The expression “liquid active agent formulation” is used herein to indicate a formulation that contains an active agent and is able to flow from the dosage form of the present invention into the environment of use. A liquid active agent formulation suitable for use in the ascending release dosage form of the present invention may be neat liquid active agent or a solution, suspension, slurry, emulsion, self-emulsifying composition, liposomal solution, or other flowable formulation in which the active agent is present. The liquid active agent formulation may be a solid, or not flowable, at temperatures lower than the temperature of the operational environment, such as the body temperature of an intended animal or human subject, but such a formulation should become flowable at least after introduction of the dosage form into the operational environment. A binder, antioxidant, pharmaceutically acceptable carrier, permeation enhancer, or the like may accompany the active agent in the liquid active agent formulation, and the liquid active agent formulation may include a surfactant of mixture of surfactants. U.S. Pat. Nos. 6,174,547 and 6,245,357 and U.S. patent applications Ser. Nos. 08/075,084, 09/733,847, 60/343,001, and 60/343,005, which are incorporated herein by reference, detail exemplary drugs, carriers, and other constituents that may be used to form a liquid active agent formulation suitable for use in the dosage form of the present invention.

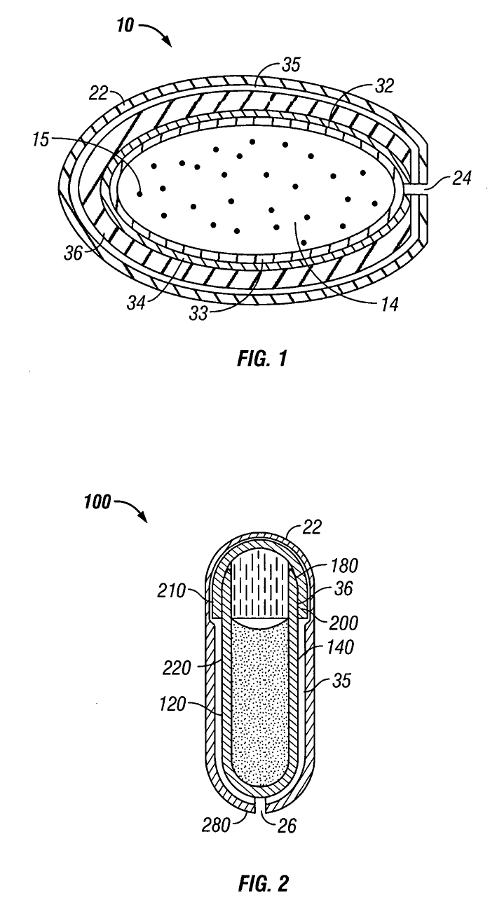

Three exemplary embodiments of a dosage form according to the present invention are illustrated in FIG. 1 through FIG. 3. In the embodiment illustrated in FIG. 1, the dosage form 10 of the present invention is formed using a soft capsule 32, or “soft-cap.” As can bee seen in FIG. 1, a barrier layer 34 is formed around the soft-cap 32, and an expandable osmotic composition 36, or “osmotic layer,” is formed around the barrier layer 34. An ascending release membrane 35 is provided around the osmotic composition 36, and a semipermeable membrane 22 is formed around the ascending release membrane 35. An exit orifice 24 is preferably formed through the semipermeable membrane 22, the ascending release membrane 35, the osmotic layer 36, and the barrier layer 34 to facilitate delivery of the liquid active agent formulation 14 from the soft-cap ascending release dosage form 10.

The soft-cap 32 used to create an ascending release dosage form 10 of the present invention may be a conventional gelatin capsule, and may be formed in two sections or as a single unit capsule in its final manufacture. Preferably, due to the presence of the barrier layer 34, the wall 33 of the soft-cap 32 retains its integrity and gel-like characteristics, except where the wall 33 dissolves in the area exposed at the exit orifice 24. Generally maintaining the integrity of the wall 33 of the soft-cap 32 facilitates well-controlled delivery of the formulation 14. However, some dissolution of portions of the soft-cap 32 extending from the exit orifice 24 during delivery of the formulation 14 may be accommodated without significant impact on the release rate or release rate profile of the liquid active agent formulation 14.

Any suitable soft-cap may be used to form an ascending release dosage form according to the present invention. The soft-cap 32 may be manufactured in accordance with conventional methods as a single body unit comprising a standard capsule shape. Such a single-body soft-cap typically may be provided in sizes from 3 to 22 minims (1 minim being equal to 0.0616 ml) and in shapes of oval, oblong, or others. The soft cap 32 may be manufactured in accordance with conventional methods using, for example, a soft gelatin material or a hard gelatin material that softens during operation. The soft cap 32 may be manufactured in standard shapes and various standard sizes, conventionally designated as (000), (00), (0), (1), (2), (3), (4), and (5), with largest number corresponding to the smallest capsule size. However, whether the soft-cap 32 is manufactured using soft gelatin capsule or hard gelatin capsule that softens during operation, the soft-cap 32 may be formed in non-conventional shapes and sizes if required or desired for a particular application.

At least during operation, the wall 33 of the soft-cap 32 should be soft and deformable to achieve a desired ascending release rate. The wall 33 of a soft-cap 32 used to create an ascending release dosage form 10 according to the present invention will typically have a thickness that is greater than the thickness of the wall of a hard capsule 120 used to create a hard capsule ascending release dosage form according to the present invention. For example, soft-caps may have a wall thickness on the order of 10-40 mils, with about 20 mils being typical, whereas hard-caps may have a wall thickness on the order of 2-6 mils, with about 4 mils being typical. U.S. Pat. Nos. 5,324,280 and 6,419,952 and U.S. applications numbered 60/343,001, and 60/343,005, the contents of which are incorporated herein by reference, describe the manufacture of various soft-caps useful for the creation of an ascending release dosage form according to the present invention.

The barrier layer 34 formed around the soft-cap 32 is deformable under the pressure exerted by the osmotic layer 36 and is preferably impermeable (or less permeable) to fluids or materials that may be present in the osmotic layer 36 and in the environment of use during delivery of the liquid active agent formulation 14. The barrier layer 34 is also preferably impermeable (or less permeable) to the liquid active agent formulation 14 of the present invention. However, a certain degree of permeability of the barrier layer 34 may be permitted if the release rate or release rate profile of the liquid active agent formulation 14 is not detrimentally affected. As it is deformable under forces applied by osmotic layer 36, the barrier layer 34 permits compression of the soft-cap 32 as the osmotic layer 36 expands. This compression, in turn, forces the liquid active agent formulation 14 from the exit orifice 24. Preferably, the barrier layer 34 is deformable to such an extent that the barrier layer 34 creates a seal between the osmotic layer 36 and the semipermeable layer 22 in the area where the exit orifice 24 is formed. In that manner, barrier layer 34 will deform or flow to a limited extent to seal the initially exposed areas of the osmotic layer 36 and the semipermeable membrane 22 when the exit orifice 24 is being formed. Materials and methods suitable for forming a barrier layer 34 included in a soft-cap controlled release dosage form 10 of the present invention are taught in U.S. Pat. No. 6,419,952 and in U.S. patent applications 60/343,001, and 60/343,005, the contents of each of which are incorporated herein by reference.

The osmotic layer 36 included in a soft-cap controlled release dosage form 10 according to the present invention includes a hydro-activated composition that expands in the presence of water or aqueous fluid, such as that present in gastric fluids. The osmotic layer 36 may be prepared using the materials and methods described in U.S. Pat. Nos. 5,324,280 and 6,419,952, and in U.S. patent application 60/392,775, the contents of each of which are herein incorporated by reference. As the osmotic layer 36 imbibes and/or absorbs external fluid, the osmotic layer 36 expands and applies a pressure against the barrier layer 34 and the wall 33 of the gel-cap 32, thereby forcing the liquid active agent formulation 14 through the exit orifice 24. The osmotic layer 36 included in a soft-cap ascending release dosage form 10 of the present invention may be configured as desired to achieve a desired release rate or delivery efficiency, and various different osmotic layer configurations that may be incorporated in an ascending release dosage form of the present invention are described in detail in U.S. Pat. Nos. 5,324,280 and 6,419,952, the contents of which incorporated herein by reference.

The semipermeable membrane 22 formed around the ascending release layer 35 is non-toxic and maintains its physical and chemical integrity during operation of the soft-cap controlled release dosage form 10. The semipermeable membrane 22 is permeable to the passage of water but is substantially impermeable to the passage of the active agent included in the liquid active agent formulation 14. Further, adjusting the thickness or material make-up of the semipermeable membrane 22 can control the maximum rate at which the osmotic layer 36 included in the dosage form 10 hydrates and expands. Therefore, the semipermeable membrane 22 coating a dosage form 10 of the present invention may be used to control the release rate achieved by the dosage form 10.

The semipermeable membrane 22 included in an ascending release dosage form 10 of the present invention may be formed using any material that is permeable to water, is substantially impermeable to the active agent, is pharmaceutically acceptable, and is compatible with the other components of the dosage form. Generally, the semipermeable membrane 22 will be formed using materials that include semipermeable polymers, semipermeable homopolymers, semipermeable copolymers, and semipermeable terpolymers. Semipermeable polymers are known in the art, as exemplified by U.S. Pat. No. 4,077,407, which is incorporated herein by this reference, and they can be made by procedures described in Encyclopedia of Polymer Science and Technology, Vol. 3, pages 325 to 354, 1964, published by Interscience Publishers, Inc., New York. The semipermeable membrane 22 included in the dosage form 10 of the present invention may also include a plasticizer to impart flexibility and elongation properties to the semipermeable membrane 22 or a flux regulating agent, such as a flux enhancing or a flux reducing agent, to assist in regulating the fluid permeability or flux through the semipermeable membrane 22. Additional references describing materials and methods suitable for fabricating the semipermeable membrane 22 included in the dosage form 10 of the present invention include, U.S. Pat. Nos. 6,174,547, 6,245,357, and 6,419,952 and U.S. patent applications Ser. Nos. 08/075,084, 09/733,847, 60/343,001, 60/343,005, and 60/392,774, the contents which are incorporated herein by reference.

It is presently preferred that a soft-cap ascending release dosage form 10 of the present invention include mechanism for sealing any portions of the osmotic layer 36 exposed at the exit orifice 24. Such a sealing mechanism prevents the osmotic layer 36 from leaching out of the system during delivery of the liquid active agent formulation 14. In one embodiment, the exit orifice 24 is drilled and the exposed portion of the osmotic layer 36 is sealed by barrier layer 34, which, because of its rubbery, elastic-like characteristics, can extend outwardly about the inner surface of exit orifice 24 during and/or after the formation of the exit orifice 24 and, in particular, as the soft-cap ascending release dosage form operates. In that manner, the barrier layer 34 effectively seals the area between the osmotic layer 34, the ascending release membrane, and the semipermeable membrane 22. In order to extend and seal, the barrier layer 34 should have an elastic, rubbery-like consistency at the temperature at which the system operation takes place. Materials, such as copolymers of ethyl acrylate and methyl methacrylate, especially Eudragit NE 30D supplied by RohmPharma, Darmstaat, Germany, are preferred. A soft-cap ascending release dosage form 10 having such a sealing mechanisms may be prepared by sequentially coating the soft-cap 32 with a barrier layer 34, an osmotic layer 36, an ascending release membrane, and a semipermeable membrane 22 and then drilling the exit orifice 24 to complete the dosage form 10. The exit orifice 24 is created through the semipermeable membrane 22, the ascending release membrane 35, the osmotic layer 36, and the barrier layer 35 to expose a portion of the soft-cap 32.

Alternatively a plug (not shown) may be used to form the desired sealing mechanism for the exposed portions of the osmotic layer 36. A plug may be formed by first providing a hole in the semipermeable membrane 22, the ascending release membrane 35, and the barrier layer 34 and then filling the hole with, for example, a liquid polymer that can be cured by heat, radiation or the like. Suitable polymers include polycarbonate bonding adhesives and the like, such as, for example, Loctite® 3201, Loctite® 3211, Loctite® 3321 and Loctite® 3301, sold by the Loctite Corporation, Hartford, Conn. Still other methods suitable for preparing a soft-cap ascending release dosage form having a seal formed on the inner surface of the exit orifice are described in U.S. Pat. Nos. 6,174,547, 6,245,357, and 6,419,952 and U.S. patent Ser. Nos. 08/075,084, 09/733,847, 60/343,001, 60/343,005.

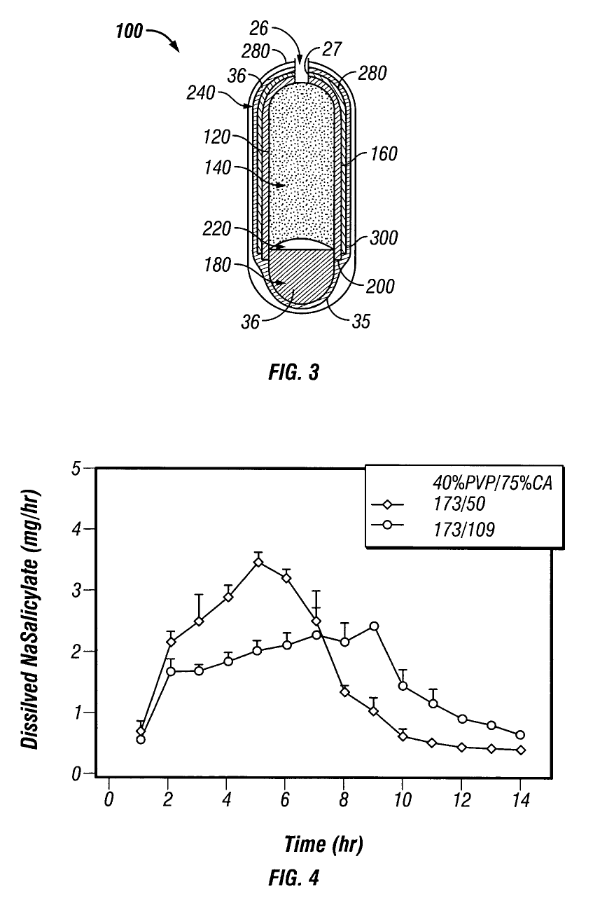

Exemplary ascending release dosage forms of the present invention manufactured using a hard capsule body 120 or “hard cap” are illustrated in FIG. 2 and FIG. 3. As can be seen in the figures, a hard-cap ascending release dosage form 100 of the present invention includes a capsule body 120 filled with a liquid active agent formulation 140, an osmotic composition 36 positioned at a first end 200 of the capsule body 120, an ascending release membrane 35 according to the present invention, and a semipermeable membrane 22 formed over the ascending release membrane 35. As is illustrated in FIG. 2 and FIG. 3, the osmotic composition 36 maybe formed as a bi-layer tableted composition having a barrier layer 220 positioned between the expandable osmotic layer 180 and the liquid active agent formulation 140. Where included, the barrier 220 layer works to prevent mixing of the liquid active agent formulation 140 with the expandable osmotic layer 180 and serves to ensure more complete delivery of the liquid active agent formulation 140 from the dosage form 100. To facilitate expulsion of the liquid active agent formulation 140, a hard-cap ascending release dosage form 100 of the present invention includes an exit orifice 260, which is preferably formed in an area near a second end 280 of the capsule body 120, with the second end being generally located opposite the osmotic composition 36.

The capsule body 120 included in the hard-cap dosage form of the present invention is formed to contain a desired amount of liquid active agent formulation 140 and includes a first end 200 and a second end 280. As can be appreciated by reference to FIG. 2 and FIG. 3, the capsule body 120 included in a hard-cap dosage form 100 of the present invention may include a cap 210, or the first end 200 of the capsule body 120 may be open, being simply sized and shaped to accommodate the osmotic composition 36. Though it is not necessary, designing the capsule body 120 to have an open first end 200 reduces contact between the osmotic composition 36 and the capsule body prior to the operation of the dosage form 100 and thereby reduces the likelihood that interaction between the expandable osmotic composition 180 and the capsule body 120 will affect the structural stability of the capsule body 120 either before or during operation of the dosage form 100. Though the capsule bodies 120 illustrated in FIG. 2 and FIG. 3 are generally oblong in shape, the capsule body of an ascending release hard-cap dosage form 100 of the present invention is not so limited and may be sized and shaped as desired to contain a desired amount of liquid active agent formulation or to suit a particular drug delivery application.

The capsule body 120 included in the hard-cap dosage form of the present invention may be formed of any suitable material. For example, the capsule body may be formed using the gelatin or polymer materials described in U.S. Pat. Nos. 6,174,547, 5,413,572 and 5,614,578 and U.S. patent application 60/392,774, which are incorporated herein by reference. In a preferred embodiment, the capsule body of a hard-cap dosage form of the present invention is formed using a water-soluble polymer material. Relative to gelatin materials typically used in capsule fabrication, water-soluble polymer materials are less susceptible to moisture loss and are markedly less sensitive to changes in moisture content. Polymer materials that can be used to form the capsule body 120 include, for example, polysaccharide materials, such as hydroxypropylmethyl cellulose (HPMC), methylcellulose, hydroxyethyl cellulose (HEC), hydroxypropyl cellulose (HPC), poly(vinylalcohol-co-ethylene glycol) and other water soluble polymers suitable for dip-coating or extrusion processes for making capsule bodies. Though the capsule body 120 included in a hard-cap dosage form 100 of the present invention may be manufactured using a single polymer material, the capsule body 120 may also be formed using a mixture of more than one polymer materials. Presently, HPMC capsules are preferably used to form the capsule body 120 of a hard-cap dosage form 100 of the present invention because HPMC capsules are commercially available and provide desirable manufacturing, stability, and delivery characteristics. The capsule body 120 of a hard-cap controlled release dosage form 100 according to the present may be formed using known manufacturing techniques, such as those described in U.S. Pat. Nos. 6,174,547, 5,413,572 and 5,614,578 and in U.S. patent application 60/392,774.

As can also be seen in FIG. 3, a hard-cap dosage form 100 of the present invention may include a water impermeable subcoat 160 formed on the capsule body 120. A water impermeable subcoat 160 works to minimize or prevent the migration of water from an external environment, through the capsule body 120, and into the liquid active agent formulation 140. In order to be effective, the water impermeable subcoat 160 need not be perfectly impermeable to the passage of water. As it is used herein, the expression “water impermeable” refers to subcoats exhibiting a water flux of less than about 10⁻⁴ (mil·cm/atm·hr). Any material that provides a subcoat of sufficient water impermeability, is pharmaceutically acceptable, and is compatible with the other components of the dosage form 100 may be used to form the water impermeable subcoat 160. However, latex materials, such as Surelease® latex materials available from Colorcon, Inc., Kollicoat (SR latex materials available from BASF, Eudragit® SR, and other polymethylacrylate latex materials, are presently preferred for forming the water impermeable subcoat 160.

The water impermeable subcoat 160 may be provided on the capsule body 120 using any suitable coating technique. For example, the capsule body 120 may be provided with a water impermeable subcoat 160 using a known dip coating process. The water impermeable subcoat 160 may also be formed over the capsule body 120 using a known spray coating process.

Where a spray coating process is used, however, and it is desired that the capsule body 120 in the finished dosage not include a cap, the capsule body 120 is preferably provided with a removable cap before the spray coating is conducted. Providing the capsule body 120 with a removable cap before the spray coating process prevents the undesirable coating of the inner surfaces of the capsule body 120 with the material forming the water impermeable subcoat 160. Moreover, where the capsule body 120 is not to include a cap 210, the spray coating process must be tailored to allow adequate coating of the water impermeable subcoat 160, while permitting removal of the removable cap after formation of the water impermeable subcoat 160 so that further processing of the coated capsule body 120 can be conducted. Such a spray coating process is described in U.S. patent application 60/392,774, the contents of which have are incorporated herein by reference.

As is true of the osmotic composition 36 included in the soft-cap dosage form 10 of the present invention, the osmotic composition 36 included in a hard-cap dosage form 100 of the present invention is formulated such that the osmotic composition 36 expands as it absorbs water from the environment of use. As the osmotic composition 36 expands, the osmotic composition 36 exerts a force against the liquid active agent formulation 140 and causes the expulsion of the liquid active agent formulation 140 through the exit orifice 26. Any composition that exhibits such characteristics, is pharmaceutically acceptable, and is compatible with the other components of the dosage form of the present invention may be used to form the osmotic composition 36 included in a hard-cap dosage form 100 of the present invention. Exemplary materials and methods for forming an expandable osmotic composition 180 for use in a hard-cap dosage form 100 of the present invention are detailed in U.S. Pat. Nos. 6,174,547 6,245,357, and 6,419,952 and in U.S. patent applications Ser. Nos. 09/733,847, 60/343,001, and 60/343,005, and 60/392,774.

As can also be appreciated by reference to FIG. 2 and FIG. 3, the osmotic composition 36 of the preferred controlled release hard-cap 100 is preferably tableted in a bi-layer tablet including an expandable osmotic layer 180 and a barrier layer 220. The barrier layer 220 works to minimize or prevent the mixing of the liquid active agent formulation 140 with the expandable osmotic layer 180 before and during operation of the dosage form 100. By minimizing or preventing mixing of the liquid active agent formulation 140 with the expandable osmotic layer 180, the barrier layer 220 serves to reduce the amount of residual active agent remaining within the dosage form 100 after the osmotic composition 36 has ceased expansion or has filled the interior of the dosage form 100. The barrier layer 220 also serves to increase the uniformity with which the driving power of the osmotic composition 36 is transferred to the liquid active agent formulation 140 included in the dosage form 100. A barrier layer 220 included in the preferred hard-cap controlled release dosage form 100 may be formed using the materials and methods described in U.S. patent applications Ser. Nos. 08/075,084, 60/343,001, 60/343,005, and 60/392,774.

The semipermeable membrane 22 included in the hard-cap dosage form 100 of the present invention is permeable to the passage of water but is substantially impermeable to the passage of the active agent included in the liquid active agent formulation 140. The semipermeable membrane 22 is non-toxic to the intended subject and maintains its physical and chemical integrity during the operation of the dosage form 100. Further, adjusting the thickness or material make-up of the semipermeable membrane 240 can control the maximum rate at which the osmotic composition 36 included in the dosage form 100 of the present invention expands. Therefore, the semipermeable membrane 22 coating the hard-cap dosage form 100 of the present invention may, in part, control the release rate or release rate profile achieved by the hard-cap dosage form 100. The semipermeable membrane 22 provided in a hard-cap controlled release dosage form of the present invention may be provided using the materials and methods already described in relation to the soft-cap controlled release dosage form 10 illustrated in FIG. 1.

The exit orifice 26 included in a hard-cap dosage form 100 of the present invention may be embodied by one of various different structures suitable for allowing the release of the liquid active agent formulation 140. As illustrated in FIG. 2 and FIG. 3, the exit orifice 26 is generally formed at or near the second end 280 of the capsule body 120 and may include an aperture 27 formed through the semipermeable membrane 22, the ascending release membrane 35, and, where provided, the water impermeable subcoat 160. The aperture 27 of the exit orifice 26 exposes a portion of the capsule body 120 but preferably does not penetrate the capsule body 120. Upon administration of the dosage form 100 to an environment of operation, water present in the environment of operation weakens or dissolves the portion of the capsule body 120 exposed by the aperture 27, allowing the liquid active agent formulation 140 contained within the capsule body 120 to be expelled. An aperture 27 used to form the exit orifice 26 shown in FIG. 2 and FIG. 3 may be simply formed using known mechanical or laser drilling techniques. Nevertheless, the hard-cap dosage form 100 of the present invention is not limited to the exit orifices 26 illustrated in FIG. 2 and FIG. 3. Further descriptions of exit orifices that may be used in a hard-cap dosage form 100 of the present are invention are described, for example, in those patents and patent applications already incorporated herein by reference, as well as in U.S. Pat. Nos. 3,845,770, 3,916,899, and 4,200,098, the contents of which are herein incorporated by this reference.

In one embodiment, a controlled release dosage form of the present invention is designed to begin release of liquid active agent formulation only after the dosage form has entered the lower GI tract of a subject. As it is used herein, the term “lower GI tract” indicates the distal small intestine and the colon of a subject. In one such embodiment, the controlled release dosage form of the present invention is provided with and enteric overcoat that works to prevent operation of the dosage form until the dosage form has entered the lower GI tract of a subject. Enteric coatings are known in the art and are designed to remain intact until exposed to an aqueous environment having a predetermined pH. Therefore, a controlled release dosage form can be according to the present invention can be provided with an enteric coating that remains intact in the upper GI tract of a subject but dissolves the in the lower GI tract due to the change in pH that occurs as the dosage form travels from the upper portions of the GI tract to the lower potions of the GI tract. Exemplary enteric coatings are discussed at, for example, Remington's Pharmaceutical Sciences, (1965), 13^{th} ed., pages 604-605, Mack Publishing Co., Easton, Pa.; Polymers for Controlled Drug Delivery, Chapter 3, CRC Press, 1991; Eudragit® Coatings Rohm Pharma, (1985); and U.S. Pat. No. 4,627,851. If desired, the thickness and chemical constituents of an enteric coating formed on a dosage form of the present invention may be selected to target release of the formulation of the present invention within a specific region of the lower GI tract.

Of course, a controlled release dosage form of the present invention designed to begin release of liquid active agent formulation after passage through the upper GI is not limited to a controlled release dosage form having an enteric coating. For instance, the semipermeable membrane, osmotic composition, or ascending release membrane may be formulated and designed such that the controlled release dosage form does not begin delivery of liquid active agent formulation for a period of time that is sufficient to generally ensure passage into the lower GI tract of the subject. Alternatively, a controlled release dosage form according to the present invention may be designed to begin delivery liquid active agent formulation in the lower GI tract of a subject by providing the dosage form with an outer coating that erodes over a desired period of time after administration, with the erosion of the coating being substantially independent of environmental pH.

Where the ascending release dosage form of the present invention is an osmotic dosage form including a semipermeable membrane, the ascending release membrane included adjacent to the semipermeable membrane is generally designed to exhibit a permeability to aqueous fluid that increases over a time to a value that is significantly larger than that exhibited by the semipermeable membrane. Such a design allows the maximum total permeability of the semipermeable membrane and ascending release membrane to be reliably determined by the maximum permeability of the semipermeable membrane and eases control of the maximum hydration rate of the osmotic composition included in the osmotic dosage form.

### EXAMPLE 1

Exemplary hard-cap ascending release dosage forms according to the present invention were manufactured, and the release rate of the dosage forms was evaluated. The exemplary hard-cap dosage forms were manufactured using a commercially available size # 0 hard capsule. The drug formulation loaded in the exemplary hard-cap dosage forms included 4 wt % Sodium Salicylate in a mixture of Cremophor EL and Myvacet 9-45. The mixture of Cremophor EL and Myvacet 9-45 included 75 wt % Cremophor EL and 25 wt % Myvacet 9-45. The drug formulation was mixed and loaded using standard techniques.

The exemplary hard-cap dosage forms were provided with tableted bi-layer osmotic compositions. The osmotic layer included in the tableted compositions was formed using 250 mg of an expandable Polyox composition, and the barrier layer was formed using 50 mg of a standard wax barrier material. The Polyox composition and wax barrier material were formed and tableted using standard methods.

The exemplary hard-cap dosage forms were coated with an ascending release membrane formed using blend of Eudragit NE and Eudragit FS combined with a cross linked polyvinylpyrrolidone (PVP XL-10). The ascending release membrane was coated using a standard spray coating process. The ascending release membrane was formulated using 40 wt % PVP XL-10 and 60 wt % of an 85/15 blend of Eudragit NE/Eudragit FS. The exemplary hard-cap dosage forms were coated with the ascending release membrane composition until an ascending release membrane of about 173 mg was achieved.

After the ascending release membrane was coated, the exemplary hard-cap dosage forms were completed by coating a semipermeable membrane over the ascending release membrane and providing each dosage form with an exit orifice. The semipermeable membrane was formed using standard coating techniques and included 75 wt % cellulose acetate 398-10 and 25 wt % Pluronic F68. However, a first batch of exemplary hard cap dosage forms was provided a relatively lighter semipermeable membrane (50 mg), while a second batch of exemplary hard-cap dosage forms was provided a relatively heavier semipermeable membrane (109 mg). After formation of the semipermeable membranes, both batches of exemplary hard-cap dosage forms were completed by providing each dosage form with a 10 mil exit orifice. The exit orifices were formed using a mechanical drill.

The exemplary hard-cap dosage forms were then placed in AIF and the release rates provided by the exemplary hard-cap dosage forms were evaluated. The results of such evaluation are shown in FIG. 4. As can be seen by reference to FIG. 4, the exemplary hard-cap dosage forms provided ascending sodium salicylate release rates, with those including the relatively heavier semipermeable membrane providing a more slowly ascending release rate, and those including the relatively lighter semipermeable membrane providing more rapidly ascending release rate.

### EXAMPLE 2

Exemplary soft-cap ascending release dosage forms according to the present invention were manufactured and the release rate of the dosage forms was evaluated. The exemplary soft-cap dosage forms were manufactured using commercially available soft capsules pre-filled with a liquid Guaifenisen formulation. The exemplary soft-caps were coated with a 37 mg barrier layer containing 50 wt % Eudragit FS and 50 wt % Eudragit NE using a standard barrier layer coating process. After formation of the barrier layer, the exemplary soft-caps were coated with 260 mg of a standard osmotic composition, and an ascending release membrane according to the present invention was provided over the osmotic composition.

The ascending release membrane included in the exemplary soft-cap dosage forms included 30 wt % PVP XL-10 and 70 wt % of an 85/15 blend of Eudragit NE/Eudragit FS. The ascending release membrane was coated over the osmotic composition using a standard spray coating process until an ascending release membrane weighing 202 mg was achieved.

After the ascending release membrane was coated, the exemplary soft-cap dosage forms were completed by coating a semipermeable membrane over the ascending release membrane and providing each dosage form with an exit orifice. The semipermeable membrane was formed using standard coating techniques and included 60 wt % cellulose acetate 398-10 and 40 wt % Pluronic F68. The exemplary soft-cap dosage forms were coated with the semipermeable membrane material until the dosage forms were coated with a semipermeable membrane weighing 108 mg. After formation of the semipermeable membranes, the exemplary soft-cap dosage forms were completed by providing each dosage form with a 38 mil exit orifice. The exit orifices were again provided using a mechanical drill.

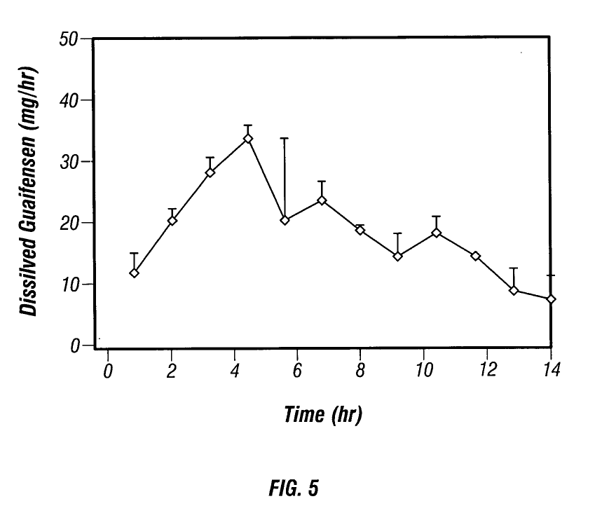

The exemplary soft-cap dosage forms were then placed in AIF and the release rates provided by the exemplary soft-cap dosage forms were evaluated. The results of such evaluation are shown in FIG. 5. As can be seen by reference to FIG. 5, the exemplary soft-cap dosage forms provided an ascending release rate of Guaifenisen over about the first 2 hours after introduction into the AIF.

We claim:

1. A dosage form comprising:
a reservoir;
a liquid active agent formulation contained within the reservoir;
an osmotic composition a semipermeable membrane;
an ascending release material adjacent to the semipermeable membrane, the ascending release material exhibiting a permeability that increases upon exposure to aqueous fluid; and
an exit orifice.

2. The dosage form of claim 1, wherein the ascending release material comprises a polymer.

3. The dosage form of claim 1, wherein the ascending release material comprises a hydrophobic polymer and a hydrophilic polymer.

4. The dosage form of claim 1, wherein the ascending release material comprises a hydrophobic polymer and a hydrophilic polymer, wherein the hydrophilic polymer is water swellable.

5. The dosage form of claim 3, wherein the hydrophobic polymer comprises about 50 wt % to about 80 wt % of the ascending release material and hydrophilic polymer comprises about 20 wt % to about 50 wt % of the ascending release material.

6. The dosage form of claim 3, wherein the hydrophobic polymer comprises about 60 wt % to about 70 wt % of the ascending release material and the hydrophilic polymer comprises about 30 wt % to about 40 wt % of the ascending release material.

7. The dosage form of claim 1, wherein the ascending release material is formulated such that the ascending release material exhibits a first permeability before exposure to an aqueous fluid and a second permeability after exposure the aqueous fluid, wherein the second permeability increases as the ascending release material is exposed to the aqueous fluid over time.

8. The dosage form of claim 1, wherein the reservoir, the osmotic composition, the semipermeable membrane, and the ascending release material adjacent to the semipermeable membrane are chosen and configured such that the dosage form provides controlled, ascending release of the liquid active agent formulation over a period of at least two hours.

9. The dosage form of claim 1, wherein the reservoir, the osmotic composition, the semipermeable membrane, and the ascending release material adjacent to the semipermeable membrane are chosen and configured such that the dosage form provides controlled, ascending release of the liquid active agent formulation over a period of about 2 to about 24 hours.

10. The dosage form of claim 1, wherein the reservoir, the osmotic composition, the semipermeable membrane, and the ascending release material adjacent to the semipermeable membrane are chosen and configured such that the dosage form provides controlled, ascending release of the liquid active agent formulation over a period of about 4 to about 12 hours.

11. The dosage form of claim 1, wherein the ascending release material comprises a hydrophobic acrylic polymer and a hydrophilic vinyl polymer.

12. The dosage form of claim 11, wherein the hydrophobic acrylic polymer comprises a 85/15 wt/wt blend of Eudragit NE/Eudragit FS and the hydrophilic vinyl polymer comprises a cross linked polyvinylpyrrolidone.

13. The dosage form of claim 1, wherein the ascending release material comprises a hydrophobic polymer and a hydrophilic polymer, and the hydrophobic polymer comprises one or more material selected from the group consisting of polystyrene, polyamides, polyvinyl acetate, poly-methylmethacrylate, ethyl acrylate methyl methacrylate copolymer, ethyl acrylate methyl methacrylate copolymer, poly(butyl methacrylate (2-dimethyl aminoethyl)methacrylate, methyl methacrylate), and methacrylic acid methylmethacrylate copolymer.

14. The dosage form of claim 1, wherein the ascending release material comprises a hydrophobic polymer and a hydrophilic polymer, and the hydrophilic polymer comprises one or more material selected from the group consisting of low substituted hydroxypropyl cellulose, hydroxypropyl methylcellulose, methylcellulose, hydroxyethyl methylcellulose, polyvinyl acetate polyvinyl pyrrolidone copolymer, gelatin, starch, polyethylene glycol polyvinyl alcohol copolymer, carrageenan, algin, agar, gum acacia, gum karyara, carob bean gum, gum tragacanth, gum ghatti guar gum, caseinates, cellulose acetate with an acetyl content of less than 20 wt %, sodium carboxymethyl cellulose, potassium carboxy methyl cellulose, polyvinyl alcohol, polyvinyl alcohol polyethylene glycol graph copolymers, cellulose acetate phthalate, hydroxypropyl methycellulose phthalate, and hydroxypropyl methyl cellulose acetate succinate.

15. A dosage form comprising:
a capsule;
a liquid active agent formulation contained within the capsule;
an osmotic composition;
a semipermeable membrane;
an ascending release material adjacent to the semipermeable membrane, the ascending release material exhibiting a permeability that increases upon exposure to aqueous fluid; and
an exit orifice.

16. A dosage form comprising:
a capsule;
a liquid active agent formulation contained within the capsule;
an osmotic composition formed around the capsule;
an ascending release material exhibiting a permeability that increases upon exposure to aqueous fluid formed over the osmotic composition;
a semipermeable membrane formed adjacent to the ascending release material; and
an exit orifice.

17. The dosage form of claim 16, wherein a barrier layer is formed around the capsule and the osmotic composition is formed around the barrier layer.

18. A dosage form comprising:
a capsule;
a liquid active agent formulation contained within the capsule;
an osmotic composition formed around the capsule;
a semipermeable membrane formed over the osmotic composition;
an ascending release material exhibiting a permeability that increases upon exposure to aqueous fluid formed over the semipermeable membrane; and
an exit orifice.

19. The dosage form of claim 18, wherein a barrier layer is formed around the capsule and the osmotic composition is formed around the barrier layer.

20. The dosage form of claim 15, wherein the capsule comprises a gelatin material.

21. The dosage form of claim 15, wherein the osmotic composition is formed of a tableted composition and is positioned at least partially within the capsule, the ascending release material is formed over the capsule and the semipermeable membrane is formed over the ascending release material.

22. The dosage form of claim 21, wherein the osmotic composition comprises a bi-layer tableted composition having an osmotic composition and a barrier layer.

23. A dosage form comprising:
a capsule;
a liquid active agent formulation contained within the capsule;
an osmotic composition formed around the capsule;
an ascending release material exhibiting a permeability that increases upon exposure to aqueous fluid, the ascending release material being formed over the osmotic composition and comprising a blend of polymers including a hydrophobic polymer and a hydrophilic polymer;
a semipermeable membrane formed adjacent to the ascending release material; and
an exit orifice.

24. The dosage form of claim 23, wherein the ascending release material comprises a water swellable hydrophilic polymer.

25. The dosage form of claim 23, wherein the hydrophobic polymer comprises about 50 wt % to about 80 wt % of the ascending release material and the hydrophilic polymer comprises about 20 wt % to about 50 wt % of the ascending release material.

26. The dosage form of claim 23, wherein the hydrophobic polymer comprises about 60 wt % to about 70 wt % of the ascending release material and the hydrophilic polymer comprises about 30 wt % to about 40 wt % of the ascending release material.

27. The dosage form of claim 23, wherein the ascending release material is formulated such that the ascending release material exhibits a first permeability before exposure to an aqueous fluid and a second permeability after exposure to the aqueous fluid, wherein the second permeability increases as the ascending release material is exposed to the aqueous fluid over time.

28. The dosage form of claim 23, wherein the capsule, the osmotic composition, the semipermeable membrane, and the ascending release material are chosen and configured such that the dosage form provides controlled, ascending release of the liquid active agent formulation over a period of at least two hours.

29. The dosage form of claim 23, wherein the capsule, the osmotic composition, the semipermeable membrane, and the ascending release material are chosen and configured such that the dosage form provides controlled, ascending release of the liquid active agent formulation over a period of about 2 to about 24 hours.

30. The dosage form of claim 23, wherein the a capsule, the osmotic composition, the semipermeable membrane, and the ascending release material are chosen and configured such that the dosage form provides controlled, ascending release of the liquid active agent formulation over a period of about 4 to about 12 hours.

31. The dosage form of claim 23, wherein the ascending release material comprises a hydrophobic acrylic polymer and a hydrophilic vinyl polymer.

32. The dosage form of claim 31, wherein the hydrophobic acrylic polymer comprises a 85/15 wt/wt blend of Eudragit NE/Eudragit FS and the hydrophilic vinyl polymer comprises a cross linked polyvinylpyrrolidone.

33. The dosage form of claim 23, wherein the hydrophobic polymer comprises one or more material selected from the group consisting of polystyrene, polyamides, polyvinyl acetate, poly-methylmethacrylate, ethyl acrylate methyl methacrylate copolymer, ethyl acrylate methyl methacrylate copolymer, poly(butyl methacrylate (2-dimethyl aminoethyl)methacrylate, methyl methacrylate), and methacrylic acid methylmethacrylate copolymer.

34. The dosage form of claim 23, wherein the hydrophilic polymer comprises one or more material selected from the group consisting of low substituted hydroxypropyl cellulose, hydroxypropyl methylcellulose, methylcellulose, hydroxyethyl methylcellulose, polyvinyl acetate polyvinyl pyrrolidone copolymer, gelatin, starch, polyethylene glycol polyvinyl alcohol copolymer, carrageenan, algin, agar, gum acacia, gum karyara, carob bean gum, gum tragacanth, gum ghatti guar gum, caseinates, cellulose acetate with an acetyl content of less than 20 wt %, sodium carboxymethyl cellulose, potassium carboxy methyl cellulose, polyvinyl alcohol, polyvinyl alcohol polyethylene glycol graph copolymers, cellulose acetate phthalate, hydroxypropyl methycellulose phthalate, and hydroxypropyl methyl cellulose acetate succinate.

35. A dosage form comprising:
a capsule body;
a liquid active agent formulation contained within the capsule body;
a tableted osmotic composition positioned at least partially within the capsule;
an ascending release material exhibiting a permeability that increases upon exposure to aqueous fluid, the ascending release material being formed over the osmotic composition and comprising a blend of polymers including a hydrophobic polymer and a hydrophilic polymer;
a semipermeable membrane formed adjacent to the ascending release material; and
an exit orifice.

36. The dosage form of claim 35, wherein the osmotic composition comprises a bi-layer tableted composition having an osmotic composition and a barrier layer.

37. The dosage form of claim 35, further comprising a water impermeable subcoat formed over an outer surface of the capsule body.

38. The dosage form of claim 37, wherein the water impermeable subcoat comprises a latex material.

39. The dosage form of claim 35, wherein the ascending release material comprises a water swellable hydrophilic polymer.

40. The dosage form of claim 35, wherein the hydrophobic polymer comprises about 50 wt % to about 80 wt % of the ascending release material and the hydrophilic polymer comprises about 20 wt % to about 50 wt % of the ascending release material.

41. The dosage form of claim 35, wherein the hydrophobic polymer comprises about 60 wt % to about 70 wt % of the ascending release material and the hydrophilic polymer comprises about 30 wt % to about 40 wt % of the ascending release material.

42. The dosage form of claim 35, wherein the ascending release material is formulated such that the ascending release material exhibits a first permeability before exposure to an aqueous fluid and a second permeability after exposure to the aqueous fluid, wherein the second permeability increases as the ascending release material is exposed to the aqueous fluid over time.

43. The dosage form of claim 35, wherein the capsule body, the osmotic composition, the semipermeable membrane, and the ascending release material are chosen and configured such that the dosage form provides controlled, ascending release of the liquid active agent formulation over a period of at least two hours.

44. The dosage form of claim 35, wherein the capsule body, the osmotic composition, the semipermeable membrane, and the ascending release material are chosen and configured such that the dosage form provides controlled, ascending release of the liquid active agent formulation over a period of about 2 to about 24 hours.

45. The dosage form of claim 35, wherein the a capsule body, the osmotic composition, the semipermeable membrane, and the ascending release material are chosen and configured such that the dosage form provides controlled, ascending release of the liquid active agent formulation over a period of about 4 to about 12 hours.

46. The dosage form of claim 35, wherein the ascending release material comprises a hydrophobic acrylic polymer and a hydrophilic vinyl polymer.

47. The dosage form of claim 35, wherein the hydrophobic acrylic polymer comprises a 85/15 wt/wt blend of Eudragit NE/Eudragit FS and the hydrophilic vinyl polymer comprises a cross linked polyvinylpyrrolidone.

48. The dosage form of claim 35, wherein the hydrophobic polymer comprises one or more material selected from the group consisting of polystyrene, polyamides, polyvinyl acetate, poly-methylmethacrylate, ethyl acrylate methyl methacrylate copolymer, ethyl acrylate methyl methacrylate copolymer, poly(butyl methacrylate (2-dimethyl aminoethyl)methacrylate, methyl methacrylate), and methacrylic acid methylmethacrylate copolymer.

49. The dosage form of claim 35, wherein the hydrophilic polymer comprises one or more material selected from the group consisting of low substituted hydroxypropyl cellulose, hydroxypropyl methylcellulose, methylcellulose, hydroxyethyl methylcellulose, polyvinyl acetate polyvinyl pyrrolidone copolymer, gelatin, starch, polyethylene glycol polyvinyl alcohol copolymer, carrageenan, algin, agar, gum acacia, gum karyara, carob bean gum, gum tragacanth, gum ghatti guar gum, caseinates, cellulose acetate with an acetyl content of less than 20 wt %, sodium carboxymethyl cellulose, potassium carboxy methyl cellulose, polyvinyl alcohol, polyvinyl alcohol polyethylene glycol graph copolymers, cellulose acetate phthalate, hydroxypropyl methycellulose phthalate, and hydroxypropyl methyl cellulose acetate succinate.

50. A method for manufacturing a dosage form providing the ascending release of an liquid active agent formulation, the method comprising:
providing a reservoir;
loading a liquid active agent formulation within the reservoir;
operatively associating an osmotic composition with the reservoir;
forming an ascending release material over at least a portion of the dosage form; and
forming a semipermeable membrane adjacent said ascending release material.
